(19) United States
(12) Patent Application Publication
(10) Pub. No.: US 2007/0232987 A1
(43) Pub. Date: Oct. 4, 2007

(54) One-hand-operated ultrasound transducer and method for delivering a controlled and uniform distribution of a sterile or a non-sterile topical reagent to skin for use in diagnostic, therapeutic, and aesthetic therapies

(76) Inventors: Vicente Diaz, New Haven, CT (US); Ivonne Ferrer Diaz, New Haven, CT (US); Alex Merkulov, Secaucus, NJ (US); Steven Milo, New York, NY (US); Alex Hung, New York, NY (US); Tom Naparst, New York, NY (US)

Correspondence Address: CHARLES E. BAXLEY, ESQ., 90 JOHN STREET, THIRD FLOOR, NEW YORK, NY, 10038, US

(21) Appl. No.: 11/708,776

(22) Filed: Feb. 21, 2007

Related U.S. Application Data
(60) Provisional application No. 60/775,626, filed on Feb. 22, 2006.

(51) Int. Cl. A61H 1/00; A61B 17/20

(52) U.S. Cl. 604/22; 601/2

(57) ABSTRACT

A one-hand-operated ultrasound transducer and method for delivering a controlled and uniform distribution of a sterile or a non-sterile topical reagent to skin for use in diagnostic, therapeutic, and aesthetic therapies. The transducer includes a head, a cartridge, and feeding apparatus for feeding contents of the cartridge out of the head. The cartridge is removably disposed in the head and delivers the controlled and uniform distribution of the sterile or the non-sterile topical reagent to the skin for use in the diagnostic, therapeutic, and aesthetic therapies.

### 1. CROSS REFERENCE TO RELATED APPLICATION

The instant non-provisional patent application claims priority from Provisional Patent No. 60/775,626 filed on Feb. 22, 2006 entitled Portable Ultrasound Device With an Internal Gel Storage and Dispensing System for Novel Gel and incorporated herein by reference thereto.

### 2. BACKGROUND OF THE INVENTION

A. Field of the Invention

The embodiments of the present invention relate to an ultrasound transducer, and more particularly, the embodiments of the present invention relate to a one-hand-operated ultrasound transducer and method for delivering a controlled and uniform distribution of a sterile or a non-sterile topical reagent to skin for use in diagnostic, therapeutic, and aesthetic therapies.

B. Description of the Prior Art

Ultrasound is sound with a frequency greater than the upper limit of human hearing. This limit being approximately 20 kilohertz, i.e., 20,000 Hertz (Hz) frequency units.

Ultrasound techniques in medicine are of growing importance because they are non-invasive, non-ionizing, and of low cost, as compared to other sensing and imaging methods. Ultrasound has an outstanding safety record, and is used for diagnostic purposes, prenatal care, therapeutic purposes, dental care, and physical therapy.

Ultrasound therapeutic applications are highly beneficial. Benign and malignant tumors and other disorders are treated using Focused Ultrasound Surgery (FUS) or High Intensity Focused Ultrasound (HIFU). More powerful ultrasound sources are used to clean teeth and generate local heating in biological tissue, i.e., in occupational therapy and cancer treatment. Extracorporeal shock wave lithotripsy uses a powerful focused ultrasound source to break up kidney stones. Ultrasound is used to deliver topical reagents transcutaneously by the process termed “sonophoresis”. Focused ultrasound sources are used for cataract treatment by phacoemulsification. Ultrasound is used in ultrasound-assisted lipectomy or liposuction. Doppler ultrasound is being tested for use in aiding tissue plasminogen activator treatment in stroke suffers. Low-intensity pulsed ultrasound is used for therapeutic tooth and bone regeneration. Ultrasound is also shown to act synergistically with antibiotics in bacterial cell killing.

Tissue causes impedance to the passage of ultrasound waves. The specific impedance of a tissue, i.e., skin, is determined by its density and elasticity. In order for the maximal transmission of energy from one medium to another, the impedance of the two media must be the same. As the impedance at the boundary between tissues increases, the amount of reflection also increases and the amount of energy transferred decreases.

The difference in impedance is greatest for the transducer/air/skin interface, which is the first boundary ultrasound waves must overcome to reach a body. To minimize the impedance at this boundary, a coupling topical reagent is utilized. Different coupling topical reagents include various skin oils, creams, and gels. If even a small air gap exists between the transducer and the skin, the proportion of ultrasound that will be reflected approaches 99.998%, which in effect means that there will be no transmission of energy into the skin.

A few benefits of controlled and uniform distribution of the coupling topical reagents on the skin include improved diagnostic imaging studies, increased effectiveness of therapeutic ultrasound techniques, and reduced topical drug doses needed for sonophoresis. Moreover, reducing topical reagents doses will likely prevent undesirable and intolerable skin side effects, such as flaking and persistent redness or burning. To date, there is no foregoing technique offering controlled and uniform distribution of topical reagents for ultrasound therapy.

The clinical application of ultrasonography by emergency and non-emergency physicians has greatly expanded over the past decade. Physicians have adopted ultrasound to advance the timely and accurate patient evaluation and treatment. Ultrasound rapidly and non-invasively defines anatomic structures and their function, provides guidance for difficult procedures, identifies pathologic conditions, and localizes sources of pain. These results are similar to those reported in studies performed in intensive care settings where ultrasound guidance for placement of internal jugular central lines has been common for years. Given the relatively low expense of ultrasound technology and its utility for procedural guidance, not using this technology in place of invasive and potentially dangerous procedures will become increasingly difficult to explain. Ultrasound-guided central line placement reduces disastrous complications, such as pneumothorax, hemothorax, arrhythmias, air embolism, and infection.

In recent years, methods improving the appearance of skin have become popular with consumers. There is at the present time a demand for cosmetic techniques reducing the outward appearance or signs of cellulite. Human skin is a composite material of the epidermis and the dermis. At the bottom of the dermis is the subcutaneous fat and reserve, i.e., deep, fat layers. The superficial fatty layer is composed of small dense pockets of fat separated by vertical fibrous septae. Cellulite develops in the superficial fat layer. No cellulite forms in the deeper fatty layer because it is loosely organized with less regular septae.

Cellulite is a cosmetic/medical condition caused by defects in the skin resulting in the skin having an “orange peel” or “cottage cheese” appearance seen in 85-98% of women of all races. Cellulite can be located in any area of the body, and is typically characterized by fat cells becoming engorged with lipids, swelling, and clumping together, as well as excess fluid retention in the dermal and subdermal regions of the skin. Ultrasound energy heats tissue, and is currently being used to treat cellulite. In addition, there are other non-invasive ultrasound devices that cavitationally lyse being used for cellulite treatment.

Attempts have been made since the 1950's to use ultrasound to deliver hydrocortisone into joint spaces or lidocaine for pain relief. Research in recent years has dramatically increased the understanding of ultrasound and its effects on skin and transport of topical reagents. Without topical compound dose and delivery regulations, ultrasound tends to cause uneven concentration and uneven drug penetration, which may lead to injuries. This is particularly true with older ultrasound equipment, but is also true of many ultrasound technology in clinical use today.

The current goal is to achieve more effective beneficial therapeutic clinical effects with topical reagents by reaching higher concentrations in the dermis without damaging the dermis and/or the epidermis. The problem has not been adequately solved by simply using topical reagents in various vehicles at higher strengths. Using solutions at higher strengths may tend to magnify the side effects. Using solutions at higher strengths may also worsen internal systemic side effects. Using solutions at higher strengths tend to work too broadly, and generally fail to selectively cause the desired effect.

Thus, there exists a need for a one-hand-operated ultrasound transducer and method for delivering a controlled and uniform distribution of a sterile or a non-sterile topical reagent to skin for use in diagnostic, therapeutic, and aesthetic therapies.

Numerous innovations for ultrasound-related devices have been provided in the prior art that will be described below, which are in chronological order to show advancement in the art, and which are incorporated herein by reference thereto. Even though these innovations may be suitable for the specific individual purposes to which they address, they each differ in structure, and/or operation, and/or purpose, from the embodiments of the present invention in that they do not teach a one-hand-operated ultrasound transducer and method for delivering a controlled and uniform distribution of a sterile or a non-sterile topical reagent to skin for use in diagnostic, therapeutic, and aesthetic therapies.

(1) U.S. Pat. No. 6,322,532 to D'Sa et al.

U.S. Pat. No. 6,322,532 issued to D'Sa et al. on Nov. 27, 2001 teaches an ultrasonic transducer operating in flexure mode providing a highly efficient and compact sonophoresis device. The device is particularly useful for efficiently enhancing permeation of a substance through a membrane, such as dermal and mucosal membranes, for purposes of transdermal/transmucosal drug delivery and/or body fluid monitoring.

(2) U.S. Pat. No. 7,004,933 to McDaniel.

U.S. Pat. No. 7,004,933 issued to McDaniel on Feb. 28, 2006 teaches a system for enhancing and improving the transcutaneous or trans dermal delivery of topical chemicals or drugs. A disposable container contains a substantially sterile unit dose of an active agent adapted for a single use in a medical treatment. The unit dose is formulated to enhance transport of the active agent through mammalian skin when the active agent is applied to the skin and the skin is exposed to light and/or ultrasound defined by at least one specific parameter.

It is apparent that numerous innovations for ultrasound-related devices have been provided in the prior art that are adapted to be used. Furthermore, even though these innovations may be suitable for the specific individual purposes to which they address, they would not be suitable for the purposes of the embodiments of the present invention as heretofore described, namely, a one-hand-operated ultrasound transducer and method for delivering a controlled and uniform distribution of a sterile or a non-sterile topical reagent to skin for use in diagnostic, therapeutic, and aesthetic therapies.

### 3. SUMMARY OF THE INVENTION

Thus, an object of the embodiments of the present invention is to provide a one-hand-operated ultrasound transducer and method for delivering a controlled and uniform distribution of a sterile or a non-sterile topical reagent to skin for use in diagnostic, therapeutic, and aesthetic therapies, which avoids the disadvantages of the prior art.

Briefly stated, another object of the embodiments of the present invention is to provide a one-hand-operated ultrasound transducer and method for delivering a controlled and uniform distribution of a sterile or a non-sterile topical reagent to skin for use in diagnostic, therapeutic, and aesthetic therapies. The transducer includes a head, a cartridge, and feeding apparatus for feeding contents of the cartridge out of the head. The cartridge is removably disposed in the head and delivers the controlled and uniform distribution of the sterile or the non-sterile topical reagent into the skin for use in the diagnostic, therapeutic, and aesthetic therapies.

The novel features considered characteristic of the embodiments of the present invention are set forth in the appended claims. The embodiments of the present invention themselves, however, both as to their construction and to their method of operation together with additional objects and advantages thereof will be best understood from the following description of the specific embodiments when read and understood in connection with the accompanying drawing.

### 4. BRIEF DESCRIPTION OF THE DRAWING

The figures of the drawing are briefly described as follows:

FIG. 1 is a diagrammatic perspective view of the one-hand-operated ultrasound transducer for delivering a controlled and uniform distribution of a sterile or a non-sterile topical reagent to skin for use in diagnostic, therapeutic, and aesthetic therapies of the embodiments of the present invention;

FIG. 2 is a reduced exploded diagrammatic perspective view of the one-hand-operated ultrasound transducer shown in FIG. 1; and

FIG. 3 is a diagrammatic cross sectional view taken along LINE 3-3 in FIG. 1.

FIG. 4 is a frontal, downward, rightward view of an ultrasonic vibrator according to the prior art.

FIG. 5 is a front elevational view of the ultrasonic vibrator of FIG. 4.

FIG. 6 is a gel container according to the prior art.

### 5. LIST OF REFERENCE NUMERALS UTILIZED IN THE DRAWING

A. General.

10 one-hand-operated ultrasound transducer of embodiments of present invention for delivering controlled and uniform distribution of sterile or non-sterile topical reagent to skin for use in diagnostic, therapeutic, and aesthetic therapies

B. Overall Configuration.

12 head

14 cartridge for delivering controlled and uniform distribution of sterile or non-sterile topical reagent to skin for use in diagnostic, therapeutic, and aesthetic therapies

16 feeding apparatus for feeding contents 18 of cartridge 14 out of head 12

18 contents of cartridge 14

C. Specific Configuration.

20 body of head 12

22 ultrasound energy source of head 12

24 cover of head 12

26 working end of body 20 of head 12

28 non-working end of body 20 of head 12

30 magnet washer of body 20 of head 12

32 interconnect end of cover 24 of head 12

34 free end of cover 24 of head 12

36 central through bore in free end 34 of cover 24 of head 12

37 sterile or non-sterile topical reagent of contents 18 of cartridge 14

38 working end of cartridge 14

40 non-working end of cartridge 14

42 screw of cartridge 14

44 plunger of cartridge 14

46 working end of screw 42 of cartridge 14

48 non-working end of screw 42 of cartridge 14

50 perforations in working end 46 of cartridge 14

52 sealing cover of cartridge 14

54 central through bore through non-working end 48 of cartridge 14

56 washer of cartridge 14

58 axial recess in non-working end 48 of screw 42 of cartridge 14

60 shaft of feeding apparatus 16

62 coupling end of shaft 60 of feeding apparatus 16

64 free end of shaft 60 of feeding apparatus 16

66 drive gear of feeding apparatus 16

68 motor gear of feeding apparatus 16

70 motor of feeding apparatus 16

72 ejecting apparatus for ejecting spent cartridge 14

74 spring of ejecting apparatus 72

76 spring washer keeper of ejecting apparatus 72

78 handle

80 buttons on handle 78

81 electronic massager contained in handle 78

82 control panel

84 wheels of control panel 82

86 LCD screen of control panel 82

### 6. DETAILED DESCRIPTION OF THE PREFERRED EMBODIMENTS

A. General.

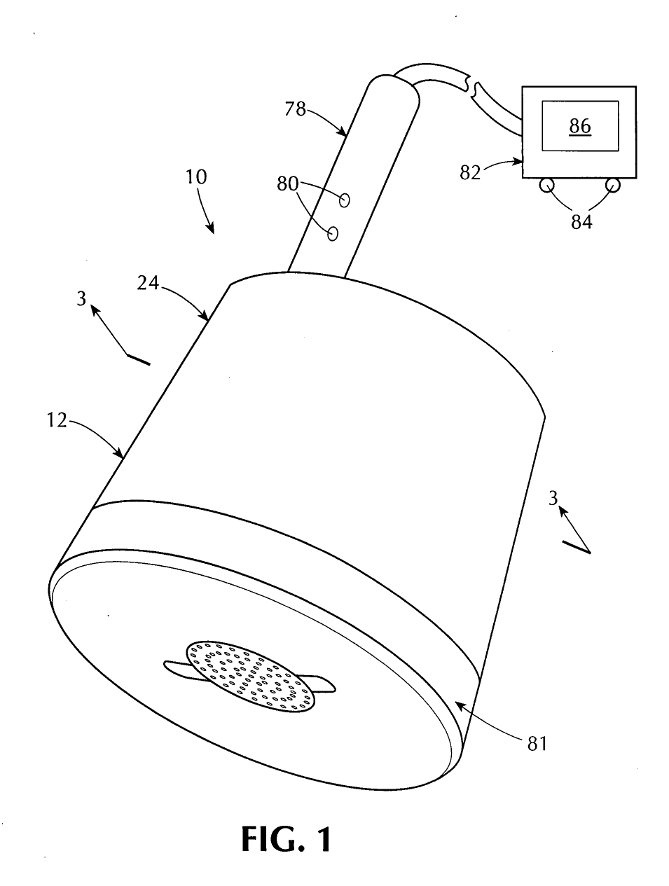

Referring now to the figures, in which like numerals indicate like parts, and particularly to FIG. 1, which is a diagrammatic perspective view of the one-hand-operated ultrasound transducer for delivering a controlled and uniform distribution of a sterile or a non-sterile topical reagent to skin for use in diagnostic, therapeutic, and aesthetic therapies of the embodiments of the present invention, the one-hand-operated ultrasound transducer of the embodiments of the present invention is shown generally at 10 for delivering a controlled and uniform distribution of a sterile or a non-sterile topical reagent to skin for use in diagnostic, therapeutic, and aesthetic therapies

B. The Overall Configuration.

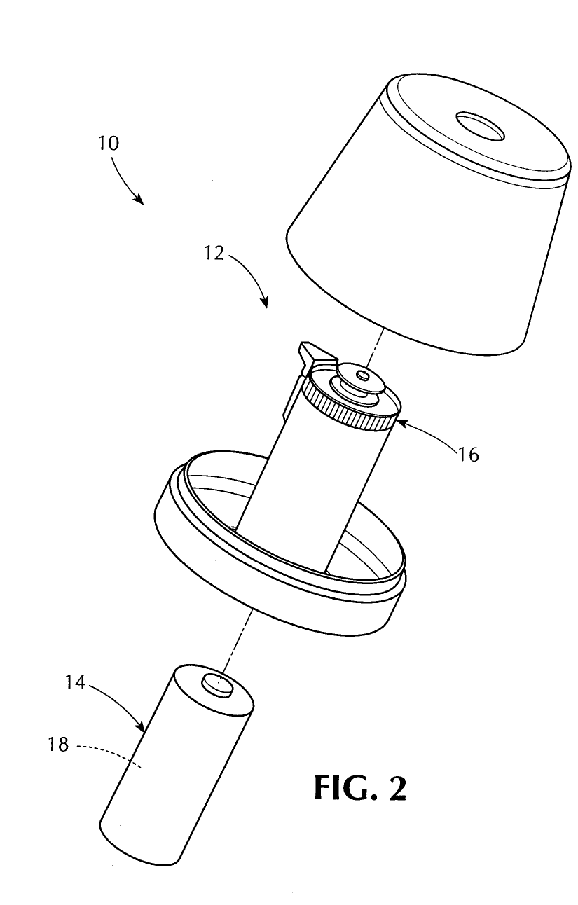

The overall configuration of the one-hand-operated ultrasound transducer 10 can best be seen in FIG. 2, which is a reduced exploded diagrammatic perspective view of the one-hand-operated ultrasound transducer shown in FIG. 1, and as such, will be discussed with reference thereto.

The one-hand-operated ultrasound transducer 10 comprises a head 12, a cartridge 14, and feeding apparatus 16 for feeding contents 18 of the cartridge 14 out of the head 12. The cartridge 14 is removably disposed in the head 12 and is for delivering the controlled and uniform distribution of the sterile or the non-sterile topical reagent to the skin for use in the diagnostic, therapeutic, and aesthetic therapies.

C. The Specific Configuration.

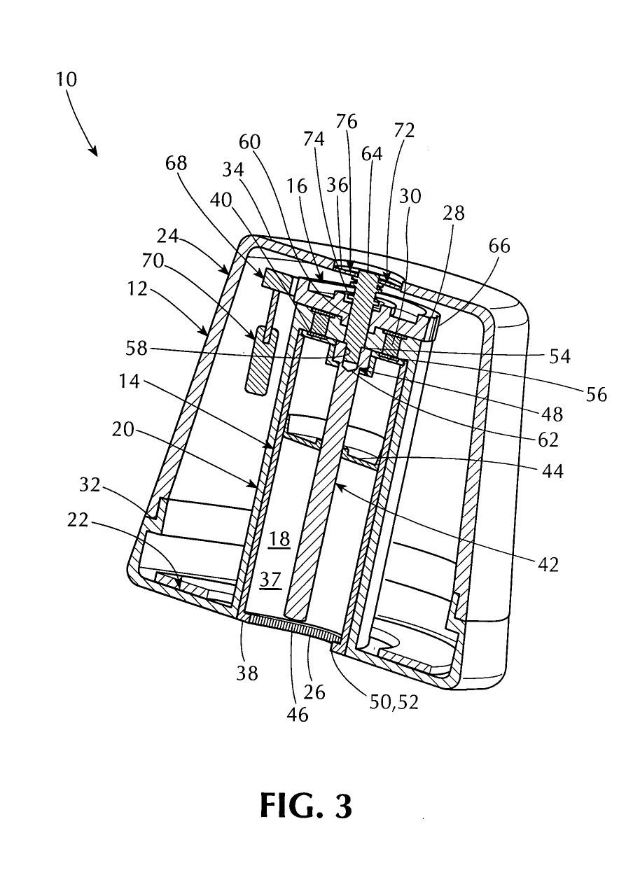

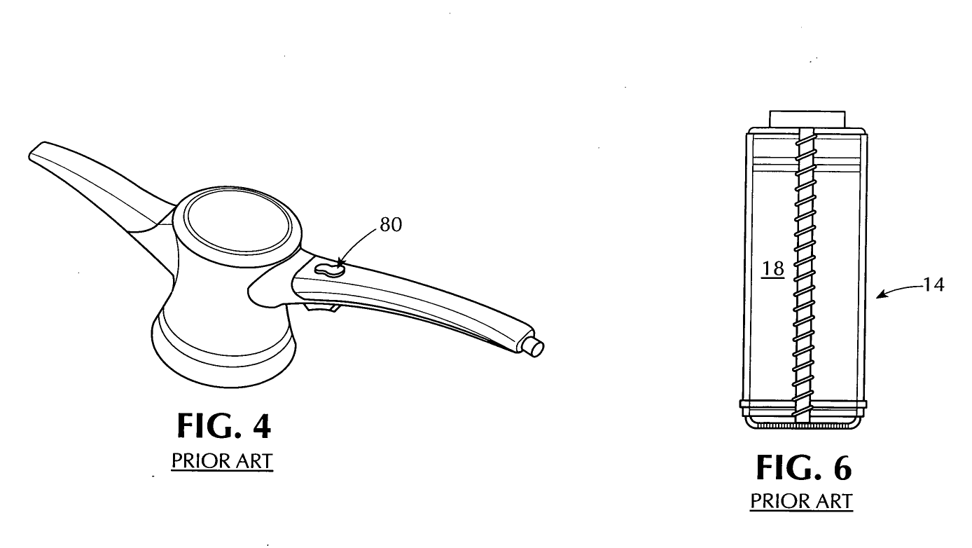

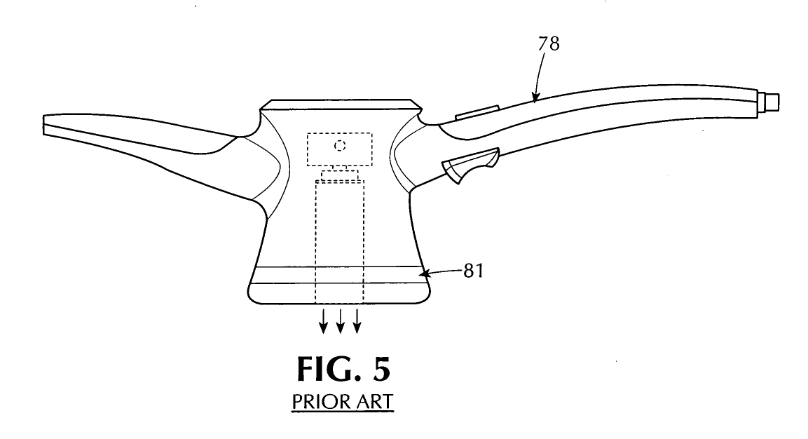

The specific configuration of the one-hand-operated ultrasound transducer 10 can best be seen in FIG. 3, which is a diagrammatic cross sectional view taken along LINE 3-3 in FIG. 1, and as such, will be discussed with reference thereto.

The head 12 comprises a body 20, an ultrasound energy source 22, and a cover 24.

The body 20 of the head 12 is hollow, generally cylindrically shaped, and has an open working end 26 and a non-working end 28 that is opposite to the working end 26 of the body 20 of the head 12 and which has a magnet washer 30 thereat.

The ultrasound energy source 22 of the head 12 is generally plate-like, surrounds the working end 26 of the body 20 of the head 12, and is a material selected from the group consisting of piezoelectric, electromagnetic, electrostrictive, electrostatic, electroscopic, magnetic, magnetostrictive, and photoacoustic transduction.

The piezoelectric material of the ultrasound energy source 22 of the head 12 is piezoceramic, which generates sound waves when an alternating electrical current is applied thereto. The sound waves have a frequency in a range of 3 KHz to 20 MHz, preferably is a resonant frequency of the transducer 10 so as to cause the transducer 10 to vibrate at its resonant frequency, are delivered in either a focused manner and concentrated to a focal point or in a non-focused manner, and have parameters further including intensity, pulse length, beam characteristics, and application time on the skin.

The cover 24 of the head 12 is generally inverted frustum-shaped, surrounds the body 20 of the head 12, and extends divergingly axially from the ultrasound energy source 22 of the head 12, at an interconnect end 32 of the cover 24 of the head 12, to past the non-working end 28 of the body 20 of the head 12, at a free end 34 of the cover 24 of the head 12.

The free end 34 of the cover 24 of the head 12 contains a central through bore 36.

The contents 18 of the cartridge 14 is a sterile or a non-sterile topical reagent 37 in a form selected from the group consisting of liquid, gel, cream, and lotion, contains a material selected from the group consisting of collagenase, aminophylline, and phosphatidylcholine, can be encapsulated in liposomes, and is exactly dosed and precisely concentrated.

The cartridge 14 is generally cylindrically shaped, removably disposed in the body 20 of the head 12, has a working end 38 and a non-working end 40 that is opposite to the working end 38 of the cartridge 14, and can be for single use.

The working end 38 of the cartridge 14 coincides with the working end 26 of the body 20 of the head 12 and dispenses the sterile or a non-sterile topical reagent 37 in the cartridge 14, and the non-working end 40 of the cartridge 14 is proximate to the non-working end 28 of the body 20 of the head 12.

The cartridge 14 further has a screw 42 and a plunger 44. The screw 42 of the cartridge 14 extends rotatably axially in the cartridge 14 from proximate to the working end 38 of the cartridge 14, at a working end 46 of the screw 42 of the cartridge 14, to the non-working end 40 of the cartridge 14, at a non-working end 48 of the screw 42 of the cartridge 14.

The plunger 44 of the cartridge 14 is disk-like, threaded onto the screw 42 of the cartridge 14, and has the sterile or a non-sterile topical reagent 37 thereinfront so as to discharge the sterile or a non-sterile topical reagent 37 when the screw 42 of the cartridge 14 rotates causing the plunger 44 of the cartridge 14 to move up the cartridge 14 and push the sterile or a non-sterile topical reagent 37 out of the working end 26 of the body 20 of the head 12 and deliver the controlled and uniform distribution of the sterile or the non-sterile topical reagent 37 to the skin for use in the diagnostic, therapeutic, and aesthetic therapies.

The working end 46 of the cartridge 14 has perforations 50 to allow the sterile or a non-sterile topical reagent 37 to exit therethrough, and which are covered by a sealing cover 52 prior to use.

The non-working end 40 of the cartridge 14 has a central through bore 54. The central through bore 54 through the non-working end 48 of the cartridge 14 is surrounded by a washer 56.

The washer 56 of the cartridge 14 is made of a ferromagnetic material so as to bond with the magnet washer 30 of the non-working end 28 of the body 20 of the head 12, and in doing so, the cartridge 14 is maintained in the body 20 of the head 12.

The non-working end 48 of the screw 42 of the cartridge 14 bulbouses out thereat, is rotatably received within the central through bore 54 in the non-working end 48 of the cartridge 14, and contains an axial recess 58.

The feeding apparatus 16 comprises shaft 60. The shaft 60 of the feeding apparatus 16 extends from replaceably couplingly in the axial recess 58 in the non-working end 48 of the cartridge 14 to rotate therewith, at a coupling end 62 of the shaft 60 of the feeding apparatus 16, to the central through bore 36 in the free end 34 of the cover 24 of the head 12, at a free end 64 of the shaft 60 of the feeding apparatus 16.

The feeding apparatus 16 further comprises a drive gear 66. The drive gear 66 of the feeding apparatus 16 is attached to the shaft 60 of the feeding apparatus 16 to rotate therewith, but is movable axially relative thereto, is disposed intermediate the coupling end 62 of the shaft 60 of the feeding apparatus 16 and the free end 64 of the shaft 60 of the feeding apparatus 16, and is adjacent to the non-working end 28 of the body 20 of the head 12.

The feeding apparatus 16 further comprises a motor gear 68. The motor gear 68 of the feeding apparatus 16 meshes with the drive gear 66 of the feeding apparatus 16 to rotate therewith.

The feeding apparatus 16 further comprises a motor 70. The motor 70 of the feeding apparatus 16 rotates the motor gear 68 of the feeding apparatus 16, and in so doing, rotates the drive gear 66 of the feeding apparatus 16, which rotates the screw 42 of the feeding apparatus 16, which causes the plunger 44 of the cartridge 14 to thread up the screw 42 of the feeding apparatus 16 and cause the sterile or the non-sterile topical reagent 37 to discharge through the working end 48 of the cartridge 14 to deliver the controlled and uniform distribution of the sterile or the non-sterile topical reagent 37 to the skin for use in the diagnostic, therapeutic, and aesthetic therapies.

The one-hand-operated ultrasound transducer 10 further comprises ejecting apparatus 72 for ejecting a spent cartridge 14.

The ejecting apparatus 72 comprises a spring 74. The spring 74 of the ejecting apparatus 72 surrounds the free end 64 of the shaft 60 of the feeding apparatus 16, and seats in the drive gear 66 of the feeding apparatus 16.

The ejecting apparatus 72 further comprises a spring washer keeper 76. The spring washer keeper 76 of the ejecting apparatus 72 is fixedly attached to the free end 64 of the shaft 60 of the feeding apparatus 16, trapping the spring 74 of the ejecting apparatus 72 against the drive gear 66 of the feeding apparatus 16. The spring washer keeper 76 of the ejecting apparatus 72 is accessible through the central through bore 36 in the free end 34 of the cover 24 of the head 12 so as to allow the shaft 60 of the ejecting apparatus 72 to be displaced inwardly moving the screw 42 of the cartridge 14 outwardly, and in doing so, ejects the cartridge 14 out of the working end 26 of the body 20 of the head 12 when the spring washer 76 of the ejecting apparatus 72 is pushed via the central through bore 36 in the free end 34 of the cover 24 of the head 12.

As shown in FIG. 1, the one-hand-operated ultrasound transducer 10 further comprises a handle 78. The handle 78 extends from the cover 24 of the head 12, has buttons 80 thereon to control delivery of the sterile or the non-sterile topical reagent 37 and activate the ultrasound energy source 22 of the head 12 with one hand, and contains an electronic massager 81.

The one-hand-operated ultrasound transducer 10 further comprises a control board 82. The control board 82 is on wheels 84, is operatively connected to the handle 78, and contains an LCD screen 86.

D. The Method.

The method of using the one-hand-operated ultrasound transducer 10 for delivering the controlled and uniform distribution of the sterile or the non-sterile topical reagent 37 to the skin for use in the diagnostic, therapeutic, and aesthetic therapies, comprises the steps of:
STEP 1: Placing the ultrasound energy source 22 of the head 12 in communication with the skin.
STEP 2: Applying an alternating electrical current to the head 12 to generate ultrasound vibrations at a particular frequency and intensity.
STEP 3: Delivering the sterile or the non-sterile topical reagent 37 from the head 12 to the skin.

When the one-hand-operated ultrasound transducer 10 is used to treat cellulite, a triple prong approach is taken, i.e., chemical, thermal, and mechanical. The chemical prong exponentially increases delivery and absorption of the sterile or the non-sterile topical reagent 37 to the skin and subcutaneous tissues. The thermal prong induces a deep tissue heat to “excite” adipocytes (fat cells) and loosen connective tissue. The mechanical prong vibrates and reshapes the dermis to reduce dimpled, puckered appearance of the skin.

E. The Conclusions.

The one-hand-operated ultrasound transducer 10:
Allows an operator to deliver sterile and non-sterile reagents to the skin under sterile or non-sterile conditions using only one hand.
Eliminates mess and necessary clean-up associated with application of reagents directly to the skin.
Allows exact and proper dosing of topical reagents.
Eliminates waste of topical reagents.
Prevents injury and side effects of improperly dosed topical reagents.
Improves quality of diagnostic images.
Improves central line placement, cellulite treatment, sonophoresis techniques, and adipose tissue lysis.

It will be understood that each of the elements described above or two or more together may also find a useful application in other types of constructions differing from the types described above.

While the embodiments of the present invention have been illustrated and described as embodied in a one-hand-operated ultrasound transducer for delivering a controlled and uniform distribution of a sterile or a non-sterile topical reagent to skin for use in diagnostic, therapeutic, and aesthetic therapies, however, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt it for various applications without omitting features that from the standpoint of prior art fairly constitute characteristics of the generic or specific aspects of the embodiments of the present invention.

The invention claimed is:

1. A one-hand-operated ultrasound transducer for delivering a controlled and uniform distribution of a sterile or a non-sterile topical reagent to skin for use in diagnostic, therapeutic, and aesthetic therapies, comprising:
a) a head;
b) a cartridge; and
c) feeding apparatus for feeding contents of said cartridge out of said head;
wherein said cartridge is removably disposed in said head; and
wherein said cartridge is for delivering the controlled and uniform distribution of the sterile or the non-sterile topical reagent to the skin for use in the diagnostic, therapeutic, and aesthetic therapies.

2. The transducer of claim 1, wherein said head comprises;
a) a body;
b) an ultrasound energy source; and
c) a cover.

3. The transducer of claim 2, wherein said body of said head is hollow; and
wherein said body of said head is generally cylindrically shaped.

4. The transducer of claim 2, wherein said body of said head has an open working end;
wherein said body of said head has a non-working end; and
wherein said non-working end of said body of said head is opposite to said working end of said body of said head.

5. The transducer of claim 4, wherein said non-working end of said body of said head has a magnet washer thereat.

6. The transducer of claim 2, wherein said ultrasound energy source of said head is generally plate-like.

7. The transducer of claim 4, wherein said ultrasound energy source of said head surrounds said working end of said body of said head.

8. The transducer of claim 2, wherein said cover of said head is generally inverted frustum-shaped.

9. The transducer of claim 5, wherein said cover of said head surrounds said body of said head; and
wherein said cover of said head extends divergingly axially from said ultrasound energy source of said head, at an interconnect end of said cover of said head, to past said non-working end of said body of said head, at a free end of said cover of said head.

10. The transducer of claim 9, wherein said free end of said cover of said head contains a central through bore.

11. The transducer of claim 1, wherein said contents of said cartridge is a sterile or a non-sterile topical reagent.

12. The transducer of claim 1, wherein said cartridge is generally cylindrically shaped.

13. The transducer of claim 10, wherein said cartridge is removably disposed in said body of said head;
wherein said cartridge has a working end;
wherein said cartridge has a non-working end; and
wherein said non-working end of said cartridge is opposite to said working end of said cartridge.

14. The transducer of claim 13, wherein said working end of said cartridge coincides with said working end of said body of said head;
wherein said working end of said cartridge dispenses said sterile or said non-sterile topical reagent in said cartridge; and
wherein said non-working end of said cartridge is proximate to said non-working end of said body of said head.

15. The transducer of claim 13, wherein said cartridge has a screw; and
wherein said screw of said cartridge extends rotatably axially in said cartridge from proximate to said working end of said cartridge, at a working end of said screw of said cartridge, to said non-working end of said cartridge, at a non-working end of said screw of said cartridge.

16. The transducer of claim 15, wherein said cartridge has a plunger; and
wherein said plunger of said cartridge is disk-like.

17. The transducer of claim 16, wherein said plunger of said cartridge is threaded onto said screw of said cartridge; and
wherein said plunger of said cartridge has said sterile or said non-sterile topical reagent thereinfront so as to discharge said sterile or said non-sterile topical reagent when said screw of said cartridge rotates causing said plunger of said cartridge to move up said cartridge and push said sterile or said non-sterile topical reagent out of said working end of said body of said head and deliver said controlled and uniform distribution of said sterile or said non-sterile topical reagent to the skin for use in the diagnostic, therapeutic, and aesthetic therapies.

18. The transducer of claim 15, wherein said working end of said cartridge has perforations; and
wherein said perforations in said working end of said cartridge allow said sterile or said non-sterile topical reagent to exit therethrough.

19. The transducer of claim 18, wherein said perforations in said working end of said cartridge are covered by a sealing cover prior to use.

20. The transducer of claim 18, wherein said non-working end of said cartridge has a central through bore.

21. The transducer of claim 20, wherein said central through bore through said non-working end of said cartridge is surrounded by a washer; and
wherein said washer of said cartridge is made of a ferromagnetic material so as to bond with said magnet washer of said non-working end of said body of said head, and in doing so, said cartridge is maintained in said body of said head.

22. The transducer of claim 20, wherein said non-working end of said screw of said cartridge bulbouses out thereat; and
wherein said non-working end of said screw of said cartridge is rotatably received within said central through bore in said non-working end of said cartridge.

23. The transducer of claim 22, wherein said non-working end of said screw of said cartridge contains an axial recess.

24. The transducer of claim 23, wherein said feeding apparatus comprises shaft; and
wherein said shaft of said feeding apparatus extends from replaceably couplingly in said axial recess in said non-working end of said cartridge to rotate therewith, at a coupling end of said shaft of said feeding apparatus, to said central through bore in said free end of said cover of said head, at a free end of said shaft of said feeding apparatus.

25. The transducer of claim 24, wherein said feeding apparatus comprises a drive gear;
wherein said drive gear of said feeding apparatus is attached to said shaft of said feeding apparatus to rotate therewith, but is movable axially relative thereto;
wherein said drive gear of said feeding apparatus is disposed intermediate said coupling end of said shaft of said feeding apparatus and said free end of said shaft of said feeding apparatus; and
wherein said drive gear of said feeding apparatus is adjacent to said non-working end of said body of said head.

26. The transducer of claim 25, wherein said feeding apparatus comprises a motor gear; and
wherein said motor gear of said feeding apparatus meshes with said drive gear of said feeding apparatus to rotate therewith.

27. The transducer of claim 26, wherein said feeding apparatus comprises a motor; and
wherein said motor of said feeding apparatus rotates said motor gear of said feeding apparatus, and in so doing, rotates said drive gear of said feeding apparatus, which rotates said screw of said feeding apparatus, which causes said plunger of said cartridge to thread up said screw of said feeding apparatus and cause said sterile or said non-sterile topical reagent to discharge through said working end of said cartridge to deliver said controlled and uniform distribution of said sterile or said non-sterile topical reagent to the skin for use in the diagnostic, therapeutic, and aesthetic therapies.

28. The transducer of claim 25, further comprising ejecting apparatus for ejecting a spent cartridge.

29. The transducer of claim 28, wherein said ejecting apparatus comprises a spring;
wherein said spring of said ejecting apparatus surrounds said free end of said shaft of said feeding apparatus; and
wherein said spring of said ejecting apparatus seats in said drive gear of said feeding apparatus.

30. The transducer of claim 29, wherein said ejecting apparatus comprises a spring washer keeper;
wherein said spring washer keeper of said ejecting apparatus is fixedly attached to said free end of said shaft of said feeding apparatus, trapping said spring of said ejecting apparatus against said drive gear of said feeding apparatus; and
wherein said spring washer keeper of said ejecting apparatus is accessible through said central through bore in said cover of said head so as to allow said shaft of said ejecting apparatus to be displaced inwardly moving said screw of said cartridge outwardly, and in doing so, ejects said cartridge out of said working end of said body of said head when said spring washer keeper of said ejecting apparatus is pushed via said central through bore in said cover of said head.

31. The transducer of claim 2, wherein said ultrasound energy source is a material selected from the group consisting of piezoelectric, electromagnetic, electrostrictive, electrostatic, electroscopic, magnetic, magnetostrictive, and photoacoustic transduction.

32. The transducer of claim 31, wherein said piezoelectric material of said ultrasound energy source of said head is piezoceramic.

33. The transducer of claim 32, wherein said piezoceramic of said piezoelectric material of said ultrasound energy source of said head generates sound waves when an alternating electrical current is applied thereto.

34. The transducer of claim 33, wherein said sound waves have a frequency in a range of 3 KHz to 20 MHz.

35. The transducer of claim 33, wherein said sound waves have a frequency that is a resonant frequency of said transducer so as to cause said transducer to vibrate at its resonant frequency.

36. The transducer of claim 33, wherein said sound waves are delivered in either a focused manner and concentrated to a focal point or in a non-focused manner.

37. The transducer of claim 1, wherein said sterile or said non-sterile topical reagent is in a form selected from the group consisting of liquid, gel, cream, and lotion.

38. The transducer of claim 1, wherein said sterile or said non-sterile topical reagent contains a material selected from the group consisting of collagenase, aminophylline, and phosphatidylcholine.

39. The transducer of claim 1, wherein said sterile or said non-sterile topical reagent is encapsulated in liposomes.

40. The transducer of claim 2, further comprising a handle; and
wherein said handle extends from said cover of said head.

41. The transducer of claim 40, wherein said handle has buttons thereon; and
wherein said buttons on said handle control delivery of said sterile or said non-sterile topical reagent and activate said ultrasound energy source of said head with one hand.

42. The transducer of claim 40, further comprising a control board; and
wherein said control board is operatively connected to said handle.

43. The transducer of claim 42, wherein said control panel is on wheels.

44. The transducer of claim 42, wherein said control panel contains an LCD screen.

45. The transducer of claim 40, wherein said handle contains an electronic massager.

46. A method of using a one-hand-operated ultrasound transducer for delivering a controlled and uniform distribution of a sterile or a non-sterile topical reagent to skin for use in diagnostic, therapeutic, and aesthetic therapies, comprising the steps of:
a) placing an ultrasound energy source of a head of the one-hand-operated ultrasound transducer in communication with the skin;
b) applying an alternating electrical current to the head to generate ultrasound vibrations at a particular frequency and intensity; and
c) delivering the sterile or the non-sterile topical reagent from the head to the skin.

47. The method of claim 46, wherein said delivering step includes delivering an exactly controlled concentration or dose of the sterile or the non-sterile topical reagent from the head to the skin.

48. The method of claim 46, further comprising when treating cellulite the steps of:
a) exponentially increasing delivery and absorption of the sterile or the non-sterile topical reagent to the skin and subcutaneous tissues;
b) inducing a combination of deep tissue heat, acoustic streaming, and micromassage to excite adipocytes and loosen connective tissue; and
c) vibrating and reshaping dermis to reduce dimpled, puckered appearance of the skin.
